(19) United States
(12) Patent Application Publication
(10) Pub. No.: US 2019/0231430 A1
(43) Pub. Date: Aug. 1, 2019

(54) FEEDBACK SYSTEM AND METHOD FOR TREATMENT PLANNING

(71) Applicant: Varian Medical Systems International AG, Cham (CH)

(72) Inventors: Anri Maarita FRIMAN, Espoo (FI); Ronan MAC LAVERTY, Helsinki (FI)

(73) Assignee: Varian Medical Systems International AG, Cham (CH)

(21) Appl. No.: 15/885,498

(22) Filed: Jan. 31, 2018

(51) Int. Cl. A61B 34/10; G06F 3/01; G06F 3/0346; G06F 3/0484; A61B 34/00; A61B 90/00

(52) U.S. Cl. CPC A61B 34/10; G06F 3/016; G06F 3/0346; G06F 3/013; G06F 3/04845; A61B 2090/502; A61B 90/37; A61B 2034/744; A61B 2034/105; A61B 2034/107; A61B 2090/366; A61B 34/76

(57) ABSTRACT

An apparatus for use in a medical process, includes: a haptic device configured to provide mechanical feedback to a user; and a processing unit communicatively coupled to the haptic device, wherein the processing unit is configured to obtain tissue information, and provide a signal to operate the haptic device based on the tissue information for assisting the user in performing treatment planning. An apparatus for use in a medical process, includes: a feedback device configured to provide visual feedback to a user; and a processing unit communicatively coupled to the feedback device; wherein the visual feedback comprises a displayed object, wherein a position of the displayed object is variable in response to operation of a user control, and wherein the processing unit is configured to obtain tissue information, and to change a behavior of the user control based on the tissue information.

### FIELD

The field of the application relates to medical devices, and more particularly, to medical devices for providing feedback for assisting a user to perform treatment planning.

### BACKGROUND

Radiation therapy involves medical procedures that selectively deliver high doses of radiation to certain areas inside a human body. Also, particle (e.g., electron, proton, etc.) beam treatment may be used to provide certain treatments. In either radiation therapy or particle beam treatment, the patient is first positioned next to the treatment machine, and a patient setup procedure is performed to align the patient with the treatment machine. After the patient has been set up, the technician then operates the treatment machine to deliver treatment energy towards the patient.

Before radiation therapy is provided to the patient, a treatment planning is first performed to create an electronic treatment plan. The treatment plan may be saved in a file, and may be processed later by a treatment machine. The treatment plan prescribes treatment parameters, such as beam delivery angles, beam energies, collimator configurations at different gantry angles, etc. When the treatment machine executes the electronic treatment plan, the treatment machine will generate treatment beams according to the treatment parameters prescribed in the treatment plan.

Currently, a treatment planning application may be employed for performing treatment planning for radiation therapy. In such treatment planning application, a user may move a cursor on a screen to select different items on a screen. However, in such treatment planning application, there is no feedback that is tied to a control that operates the cursor.

New devices and methods for providing feedback to assist a user in performing treatment planning are described herein.

### SUMMARY

An apparatus for use in a medical process, includes: a haptic device configured to provide mechanical feedback to a user; and a processing unit communicatively coupled to the haptic device, wherein the processing unit is configured to obtain tissue information, and provide a signal to operate the haptic device based on the tissue information for assisting the user in performing treatment planning.

Optionally, the apparatus further includes a non-transitory medium storing movement-vs-intensity profiles for different types of tissue, wherein the tissue information is associated with one of the types of tissue.

Optionally, one of the movement-vs-intensity profiles indicates how an intensity of force resistance, or an intensity of vibration, changes with user movement.

Optionally, the different types of tissue comprise two or more of bladder, spine, liver, kidney, cochlea, target, and critical organ.

Optionally, the haptic device is configured to provide force resistance as the mechanical feedback.

Optionally, an intensity of the force resistance is variable in correspondence with the tissue information.

Optionally, the haptic device is configured to provide vibration as the mechanical feedback.

Optionally, an intensity of the vibration is variable in correspondence with the tissue information.

Optionally, the tissue information comprises a type of tissue, a position of the tissue, clinical information about the tissue, a radiological property of the tissue, or any combination of the foregoing.

Optionally, the haptic device comprises a stick for held by the user.

Optionally, the haptic device comprises a mouse.

Optionally, the haptic device comprises a touch screen.

Optionally, the processing unit is configured to provide the feedback for assisting the user in performing structure contouring.

Optionally, the processing unit is configured to provide the feedback for assisting the user in performing dose painting.

Optionally, the apparatus further includes a wearable device with a screen, the screen being communicatively coupled to the processing unit.

Optionally, the apparatus further includes an orientation sensor coupled to the wearable device, wherein the processing unit is configured to vary an object displayed on the screen based on an input from the orientation sensor.

Optionally, the apparatus further includes a positioning device coupled to the wearable device, wherein the processing unit is configured to vary an object displayed on the screen based on an input from the positioning device.

Optionally, the wearable device comprises a virtual-reality device.

Optionally, the screen comprises a transparent screen for allowing the user to see surrounding space.

Optionally, the apparatus further includes a device with a screen, the screen being communicatively coupled to the processing unit.

Optionally, the screen is a part of a handheld device.

Optionally, the processing unit is configured to cause the screen to display an object, and to vary a configuration of the object in correspondence with a viewing direction of the user.

An apparatus for use in a medical process, includes: a feedback device configured to provide visual feedback to a user; and a processing unit communicatively coupled to the feedback device; wherein the visual feedback comprises a displayed object, wherein a position of the displayed object is variable in response to operation of a user control, and wherein the processing unit is configured to obtain tissue information, and to change a behavior of the user control based on the tissue information.

Optionally, the feedback comprises a screen.

Optionally, the apparatus further includes a non-transitory medium storing movement-vs-intensity profiles for different types of tissue, wherein the tissue information is associated with one of the types of tissue.

Optionally, one of the movement-vs-intensity profiles indicates how an intensity of force resistance, or an intensity of vibration, changes with user movement.

Optionally, the different types of tissue comprise two or more of bladder, spine, liver, kidney, cochlea, target, and critical organ.

Optionally, the tissue information comprises a type of tissue, a position of the tissue, clinical information about the tissue, a radiological property of the tissue, or any combination of the foregoing.

Optionally, the operation of the user control is for performing structure contouring.

Optionally, the operation of the user control is for performing dose painting.

Optionally, the processing unit is configured to change the behavior of the user control by changing an amount of movement of the displayed object per unit of user movement on the user control.

A method for treatment planning, includes: receiving an input from a haptic device for moving an object in a screen; obtaining tissue information by a processing unit; and generating a signal by the processing unit to operate the haptic device based on the tissue information to assist a user in performing treatment planning.

A method for treatment planning, includes: receiving an input from a user control for moving an object in a screen; obtaining tissue information by a processing unit; and changing a behavior of the user control based on the tissue information to assist a user in performing treatment planning.

Other and further aspects and features will be evident from reading the following detailed description.

### DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only exemplary embodiments and are not therefore to be considered limiting in the scope of the claims.

FIG. 1 illustrates a treatment system.

FIG. 2 illustrates an apparatus for use in a medical process.

FIGS. 3A-3F illustrate examples of movement-vs-intensity profiles for different types of tissue.

FIG. 4A illustrates an apparatus for use in a medical process.

FIG. 4B illustrates an implementation of the apparatus of FIG. 4A.

FIG. 5A-5B illustrate an example of the apparatus providing a graphical representation of medical information in an overlay configuration with respect to a patient or an image of the patient.

FIG. 5C illustrates what the user will see without the benefit of the apparatus of FIG. 5A.

FIGS. 6A-6B illustrates another example of the apparatus providing a graphical representation of medical information in an overlay configuration with respect to a patient or an image of the patient.

FIG. 6C illustrates what the user will see without the benefit of the apparatus of FIG. 6A.

FIG. 7 illustrates a method in accordance with some embodiments.

FIG. 8 illustrates another method in accordance with some embodiments.

FIG. 9 illustrates a specialized processing system.

### DETAILED DESCRIPTION

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

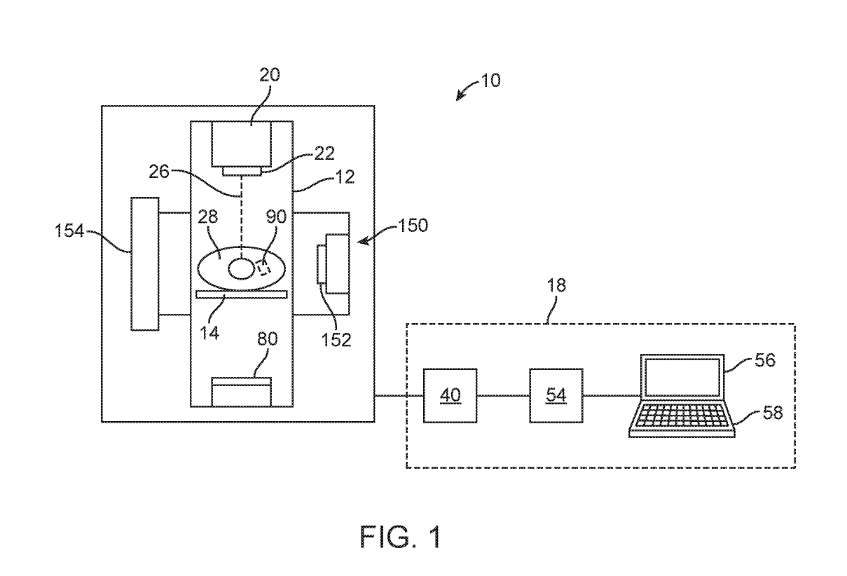

FIG. 1 illustrates a radiation system 10. The system 10 is a treatment system that includes a gantry 12, a patient support 14 for supporting a patient 28, and a control system 18 for controlling an operation of the gantry 12. The gantry 12 is in a form of an arm, but in other embodiments, the gantry 12 may have other forms (such as a ring form, etc.). The system 10 also includes a radiation source 20 that projects a beam 26 of radiation towards a patient 28 while the patient 28 is supported on support 14, and a collimator system 22 for controlling a delivery of the radiation beam 26. The collimator 22 may be configured to adjust a cross sectional shape of the beam 26. The radiation source 20 can be configured to generate a cone beam, a fan beam, or other types of radiation beams in different embodiments.

As shown in the figure, the system 10 also includes an imager 80, located at an operative position relative to the source 20 (e.g., under the support 14). In the illustrated embodiments, the radiation source 20 is a treatment radiation source for providing treatment energy. In such cases, the treatment energy may be used to obtain images. In order to obtain imaging using treatment energies, the imager 80 is configured to generate images in response to radiation having treatment energies (e.g., MV imager). In other embodiments, in addition to being a treatment radiation source, the radiation source 20 can also be a diagnostic radiation source for providing diagnostic energy for imaging purpose. In further embodiments, the system 10 may include the radiation source 20 for providing treatment energy, and one or more other radiation sources for providing diagnostic energy. In some embodiments, the treatment energy is generally those energies of 160 kilo-electron-volts (keV) or greater, and more typically 1 mega-electron-volts (MeV) or greater, and diagnostic energy is generally those energies below the high energy range, and more typically below 160 keV. In other embodiments, the treatment energy and the diagnostic energy can have other energy levels, and refer to energies that are used for treatment and diagnostic purposes, respectively. In some embodiments, the radiation source 20 is able to generate X-ray radiation at a plurality of photon energy levels within a range anywhere between approximately 10 keV and approximately 20 MeV. In other embodiments, the radiation source 20 may be configured to generate radiation at other energy ranges.

In the illustrated embodiments, the control system 18 includes a processing unit 54, such as a computer processor, coupled to a control 40. The control system 18 may also include a monitor 56 for displaying data and an input device 58, such as a keyboard or a mouse, for inputting data. The operation of the radiation source 20 and the gantry 12 are controlled by the control 40, which provides power and timing signals to the radiation source 20, and controls a rotational speed and position of the gantry 12, based on signals received from the processing unit 54. In some cases, the control 40 may also control the collimator system 22 and the position of the patient support 14. Although the control 40 is shown as a separate component from the gantry 12 and the processor 54, in alternative embodiments, the control 40 can be a part of the gantry 12 or the processing unit 54.

In the illustrated embodiments, the system 10 also includes an imaging device 150 having an imaging source 150 and an imager 154. The imaging device 150 is configured to obtain one or more images of an internal part of the patient 28. The image(s) obtained by the imaging device 150 may be used to monitor a position of the patient 28. In some cases, the imaging device 150 may be configured to obtain images of an internal fiducial 90 of the patient 28. The internal fiducial 90 may be an internal structure inside the patient 28. In some embodiments, the internal structure may move in correspondence (e.g., in sync) with a target of the patient 28 that is desired to be treated. In such cases, the internal structure may be used as a surrogate for determining a position and/or movement of the target during treatment of the patient 28, and motion management based on the surrogate may be employed in some cases. Thus, the internal fiducial 90 may be imaged by the imaging device 150 (or radiation source 20 and imager 80) that functions as a position monitoring system during a treatment of the patient 28. By means of non-limiting examples, the internal fiducial 90 may be an anatomical surrogate, such as bony structure, a vessel, a natural calcification, or any other items in a body.

In some embodiments, the imaging device 150 may be a x-ray device. In such cases, the imaging source 150 comprises a radiation source. In other embodiments, the imaging device 150 may have other configurations, and may be configured to generate images using other imaging techniques. For example, in other embodiments, the imaging device 150 may be an ultrasound imaging device, a MRI device, a tomosynthesis imaging device, or any of other types of imaging devices. Also, in the above embodiments, the imaging device 150 is illustrated as being integrated with the treatment machine. In other embodiments, the imaging device 150 may be a separate device that is separate from the treatment machine. In addition, in some embodiments, the imaging device 150 may be a room-based imaging system or a couch based imaging system. In either case, the imaging device 150 may provide any form of imaging, such as x-ray imaging, ultrasound imaging, MRI, etc. Furthermore, in other embodiments, the imaging device 150 may provide in-line imaging in the sense that it may be configured to acquire images along the same direction as the treatment beam. For example, a dual-energy source may be provided to provide imaging energy for generating an image, and to provide treatment energy to treat a patient along the same direction. In still further embodiments, the imaging device 150 may be configured to provide dual energy imaging and any form of energy-resolved imaging to increase contrast in x-ray images. For example, a first part of an image may be generated using a first energy, and a second part (e.g., a more relevant part that includes a target) of the same image may be generated using a second energy that is higher than the first energy. As a result, the second part of the image will have higher contrast compared to the first part. However, the overall dose involved in generating the whole image may be reduced compared to the situation in which the entire image is generated using the second energy.

Before the system 10 is used to treat the patient 28, a treatment plan is first determined for the patient 28. For example, a technician may obtain a treatment plan image of the patient 28, and may process the treatment plan image to create the treatment plan. By means of non-limiting examples, the treatment plan image may be a CT image, a PET-CT image, a SPECT-CT image, a x-ray image, an ultrasound image, a MRI image, a tomosynthesis image, etc. When creating the treatment plan, a treatment plan software (application) may be utilized to assist the technician to create the treatment plan. For example, the technician may use the treatment plan software to delineate anatomical structures (target and critical organs) in the patient 28, and determine different beam delivery angles for delivering treatment energies towards the target while minimizing delivery of the energies to the critical organs. The user may also use the treatment plan software to create constraints (e.g., minimum dose to be delivered to the target, maximum allowable dose for critical organs, etc.) for the treatment planning. The treatment plan may be stored as an electronic file, and may be retrieved by the system 10 later.

On the day of the treatment, the system 10 retrieves the stored treatment plan (e.g., from a medium), and processes the treatment plan to deliver treatment energies towards the target in the patient 28. For example, a processor of the system 10 may electronically process the treatment plan to activate one or more components of the system 10 to deliver the treatment energy. The processor of the system 10 may cause the gantry 12 to rotate to a certain gantry angle prescribed by the treatment plan, and to deliver certain amount of treatment energy from the gantry angle towards the target in the patient 28. The processor of the system 10 may also control the collimator 22 to shape the beam 26 while the energy source 20 is at the gantry angle. The treatment plan may prescribe that treatment energies be delivered from multiple gantry angles. Also, the treatment plan may prescribe that the patient be treated multiple times on multiple days.

The radian treatment may include multiple fractions, and it is desirable that the radiation is delivered to the correct spot in all of the fractions. In some cases, the daily situation at the time of treatment delivery might differ considerably from the situation predicted in the treatment plan, due to, for examples, internal organ movement (e.g., bladder filling, bowel movement, etc.), patient weight loss, tumor shrinkage, etc. In certain occasions, if the difference between the actual situation at the time of treatment delivery and the predicted situation in the treatment plan is too great, the goal of the treatment may no longer be met. In such cases, a new treatment plan is needed. In one implementation, for each treatment fraction (or every m fractions) a kV or cone beam CT (CBCT) is taken, and the current patient geometry is analyzed by visual inspection. Based on knowledge and assessment of the situation, the staff then decides if the patient needs a new re-plan or if the current plan is good enough. If re-plan is needed, the staff may then use the treatment planning software to perform a re-planning to determine a new treatment plan.

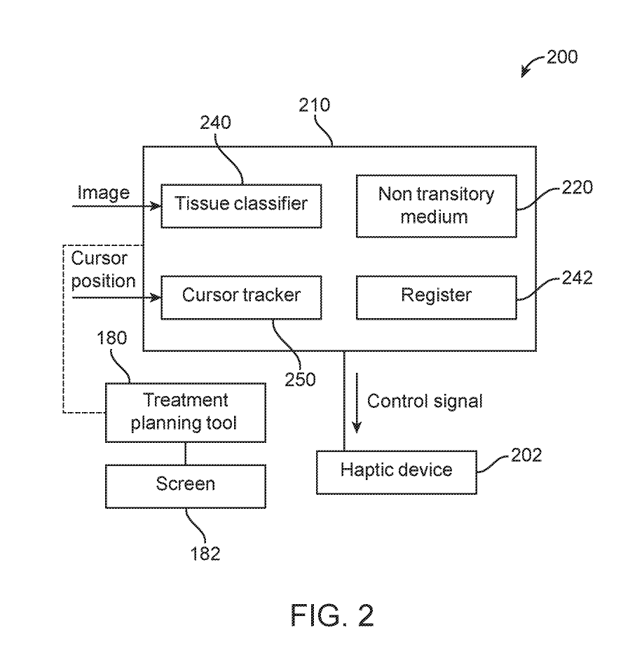

FIG. 2 illustrates an apparatus 200 for use in a medical process. The apparatus 200 is configured for providing user feedback, and is also configured to cooperate with a treatment planning tool (e.g., a treatment planning software/application) 180. In some cases, the apparatus 200 may also include the treatment planning tool 180. The apparatus 200 includes a haptic device 202 configured to provide mechanical feedback to a user; and a processing unit 210 communicatively coupled to the haptic device 202. As shown in the figure, the treatment planning tool 180 is configured to communicatively couple with a screen 182 for providing a user interface, which allows a user to perform treatment planning tasks. Alternatively, or additionally, the processing unit 210 may also be communicatively coupled to the screen 182. In one implementation, the treatment planning tool 180 may be integrated with, or included in, the processing unit 210.

The haptic device 202 may be any device that is capable of providing force feedback to the user. By means of non-limiting examples, the haptic device 202 may be one or more haptic gloves for worn by the user, a stick for held by the user, a mouse, a touch screen, a wrist band, etc.

The treatment planning tool is configured to provide a user interface for allowing a user to perform treatment planning tasks. The user interface may be displayed on a screen 182, and may be configured to provide an image of a patient, and one or more tools for allowing a user to create a treatment plan based on the image of the patient. For example, the user may operate a user control (e.g., a mouse, a touch pad, etc.) to move a cursor on the screen 182 to different parts of the image of the patient. The user may also perform structure contouring, segmentation, dose painting, or any combination of the foregoing, at different parts of the image of the patient.

The processing unit 210 is configured to track a position of the cursor in the screen 182, and provide feedback to the user based on a positioning of the cursor. In the illustrated embodiments, the processing unit 210 is configured to obtain tissue information (e.g., type of tissue at which the cursor is positioned in the image of the patient), and provide a signal to operate the haptic device 202 based on the tissue information for assisting the user in performing treatment planning. For example, when the cursor in the screen 182 is positioned over a bladder region in the image, the processing unit 210 is configured to operate the haptic device 202 to provide a first type of feedback to the user to indicate that the cursor is at a bladder region. When the cursor is positioned over a liver region in the image, the processing unit 210 is configured to operate the haptic device 202 to provide a second type of feedback to the user to indicate that the cursor is at a liver region. In some embodiments, the processing unit 210 may be communicatively coupled to the haptic device 202 via one or more wires. In other embodiments, the processing unit 210 may be communicatively coupled to the haptic device 202 via a wireless communication component.

In the illustrated embodiments, the apparatus 200 further includes a non-transitory medium 220 storing movement-vs-intensity profiles for different types of tissue. The processing unit 210 may be configured to retrieve one of the movement-vs-intensity profiles, and operate the haptic device 202 based on the retrieved movement-vs-intensity profile. In some embodiments, the processing unit 210 may be configured to use data in the retrieved profile as the tissue information, and operate the haptic device 202 based on such tissue information. Alternatively, a tissue type that is associated with the retrieved movement-vs-intensity profile may be considered as an example of tissue information, based on which the processing unit 210 is configured to operate the haptic device 202.

In other embodiments, instead of being a part of the processing unit 210, the non-transitory medium 220 may be outside the processing unit 210. In further embodiments, instead of being a part of the apparatus 200, the non-transitory medium 220 may be outside and separate from the apparatus 200. In such cases, the processing unit 210 of the apparatus 200 may be configured to communicate with the non-transitory medium 220 via a cable or wireless communication component.

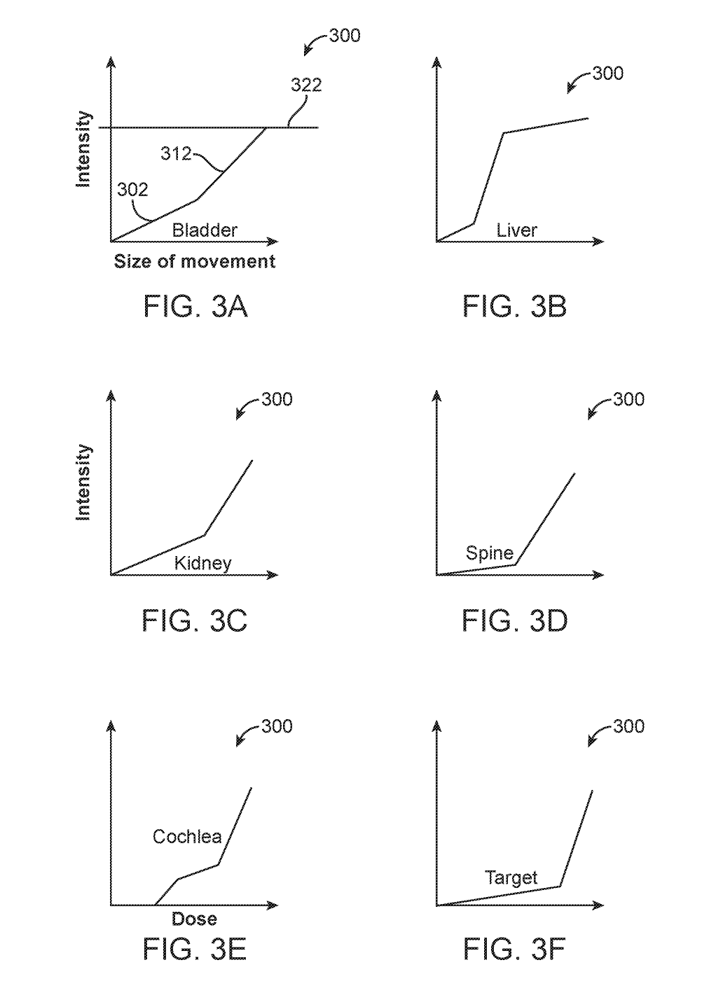

A movement-vs-intensity profile is configured to indicate how an intensity of user feedback (e.g., force resistance, vibration, etc.) changes with user movement (movement of user control). FIGS. 3A-3F illustrate examples of movement-vs-intensity profiles for different types of tissue. As shown in FIG. 3A, the movement-vs-intensity profile 300 for bladder may have a first section 302 with a first slope, which governs how intensity varies with control movement of the haptic device 202. The profile 300 may also have a second section 312 with a second slope, which governs how intensity varies with control movement of the haptic device 202 when the movement size is above a certain threshold (e.g., first threshold). The profile 300 may also have a third section 322 with a third slope, which governs how intensity varies with control movement of the haptic device 202 when the movement size is above a certain threshold (e.g., second threshold).

It should be noted that the movement-vs-intensity profiles are not limited to the examples described, and that a movement-vs-intensity profile may have other configurations in other embodiments. For example, in other embodiments, a movement-vs-intensity profile may have a curvilinear profile. In further embodiments, a movement-vs-intensity profile may have a non-continuous profile (e.g., having discrete points, or step-wise configuration). In still further embodiments, the movement-vs-intensity profile may not have the data structure (e.g., (movement, intensity)) described, and may instead be just a single intensity value. For example, different types of tissue may have different respective intensity values (for intensity of feedback).

As can be seen from the examples of FIGS. 3A-3F, the movement-vs-intensity profiles 300 are different for the different types of tissue. This allows the haptic device 202 to provide different “feel” for the user, depending on the position at which the user is operating the user control. For example, when the user is operating a cursor when the cursor is over a region of an image that corresponds with liver, the processing unit 210 may then select the movement-vs-intensity profile for the liver (i.e., the profile 300 of FIG. 3B in the example) for providing feedback to the user. On the other hand, if the cursor is over a region of the spine in the image, the processing unit 210 may select the movement-vs-intensity profile for the spine (i.e., the profile 300 of FIG. 3D in the example) for providing feedback to the user. Therefore, as the user navigates the cursor across an image that has different tissue type, the feedback input provided to the user through the haptic device 202 will be different. This allows the apparatus 200 to inform the user of the different tissue type through mechanical feedback while the user is moving the cursor across different types of tissue in the screen 182.

Returning to FIG. 2, in one implementation, the processing unit 210 may include a tissue classifier 240 for analyzing an image in order to identify different types of tissue at different locations in the image. The tissue classifier 240 may include an image analyzer for identifying different types of tissue based on shapes and/or profiles of the structures in the image. The image analyzer may also identify different types of tissue based on features' locations. For example, the liver is generally located at a certain position with respect to the lung. Also, the liver generally has a triangular profile. As such, the image analyzer may be configured to look for a triangular structure below the lung to identify the liver. The processing unit 210 may also include a register 242 for registering or associating the different identified tissue type with corresponding movement-vs-intensity profiles 300 stored in the non-transitory medium 220. For example, once a region in an image has been identified as a liver image, the register 242 may then register such region of the image with the movement-vs-intensity profile for the liver (like that shown in FIG. 3B) stored in the non-transitory medium 220. Thus, an image of the patient displayed in the screen 182 may have different regions registered with different respective movement-vs-intensity profiles 300.

Returning to FIG. 2, the processing unit 210 may further include a cursor tracker 250 for tracking a position of the cursor in an image. If the cursor tracker 250 determines that the cursor is at a liver region in an image, the processing unit 210 may then apply the movement-vs-intensity profile for the liver that is registered with the liver region of the image for providing feedback to the user.

In some embodiments, the non-transitory medium 220 may store only one movement-vs-intensity profile. For example, the movement-vs-intensity profile may be that for the target tissue. In such cases, the apparatus 200 will apply the movement-vs-intensity profile of the target tissue only when the cursor is at the target tissue in the image. When the cursor is not at the target tissue, the apparatus 200 will not apply any movement-vs-intensity profile, or may apply a default profile (e.g., which represents the situation in which no mechanical feedback is provided to the user). In other embodiments, the non-transitory medium 220 may store at least two movement-vs-intensity profile for at least two different types of tissue. The different types of tissue may be two or more of bladder, spine, liver, kidney, cochlea, target, and critical organ.

In the illustrated embodiments, the haptic device 202 is configured to provide force resistance as the mechanical feedback. In such cases, an intensity of the force resistance may be variable in correspondence with the tissue information (e.g., type of tissue) and/or a position of a cursor.

In other embodiments, the haptic device 202 may be configured to provide vibration as the mechanical feedback. In such cases, an intensity of the vibration is variable in correspondence with the tissue information (e.g., type of tissue) and/or a position of a cursor.

It should be noted that the tissue information obtained by the processing unit 210 is not limited to the examples described, and that the tissue information (based on which mechanical feedback is provided) may be any of other data. By means of non-limiting examples, the tissue information may be a type of tissue, a position of the tissue, clinical information about the tissue, a radiological property of the tissue, or any combination of the foregoing.

In some embodiments, the processing unit 210 may be configured to provide feedback for assisting the user in performing any task(s) involved in treatment planning. By means of non-limiting examples, the task(s) may include, structure contouring, segmentation, dose painting, or any combination of the foregoing. Also, the treatment planning may be for determining a treatment plan for radiotherapy, particle beam treatment (e.g., proton beam treatment), ultrasound energy treatment, or any of other types of medical treatment. As used in this specification, the term “treatment planning” refers to any process, task, or action that may affect an outcome of a treatment. Such process, task, or action may be performed before treatment energy is delivered to the patient, while treatment energy is being delivered, or between deliveries of treatment energies. Such process, task, or action may be performed in a day that is different from the treatment day. Alternatively, such process, task, or action may be performed on the same day as the treatment day (e.g., while the patient is being supported on a patient support in a treatment room).

In the above embodiments, the treatment planning tool 180 was described as providing a user interface for display on the screen 182 for presenting image and treatment planning parameters to a user. In some cases, the screen 182 may be considered to be a part of the treatment planning tool 180 and/or the apparatus 200. The screen 182 may be a computer screen, a laptop screen, a panel, a TV screen, an IPAD screen, IPAD MINI screen, a tablet screen, an IPHONE screen, a smart phone screen, or a part of any of other types of handheld devices.

In the above embodiments, the apparatus 200 was described as having a haptic device 202 for providing mechanical feedback for a user. In other embodiments, the apparatus 200 may not include the haptic device 202. Instead, the apparatus 200 may utilize the display 182 to provide visual feedback that “simulates” resistance-to-movement visually. The processing unit 210 may be configured to cause the screen to display an object, such as a cursor. The object's position in the screen is variable in response to operation of a user control, such as a mouse, a touchpad, a joy stick, a touch dome, etc. In the illustrated embodiments, the processing unit 210 is configured to obtain tissue information, and to change a behavior of the user control based on the tissue information. For example, if the cursor is being operated over a part of an image that belongs to a target, the cursor control may have a relatively higher sensitivity to control movement (e.g., a unit of control movement applied on a user control may result in the cursor moving three units in the screen). On the other hand, if the cursor is being operated over a part of an image that belongs to a bladder, the cursor control may have a relatively lower sensitivity to control movement (e.g., a unit of control movement applied on a user control may result in the cursor moving one unit in the screen). In some embodiments, the movement-vs-intensity profiles 300 described previously with reference to FIGS. 3A-3F are also applicable for providing virtual resistance as feedback for the user. When applied for providing virtual resistance, the intensity at the vertical axis of the profile 300 represents an intensity of the virtual resistance. The higher the intensity value, the less sensitivity (i.e., less cursor movement per unit of user control movement) of the cursor control is provided to the user.

In the above embodiments, the screen 182 may be a computer screen, a laptop screen, a panel, a TV screen, an IPAD screen, IPAD MINI screen, a tablet screen, an IPHONE screen, a smart phone screen, or a part of any of other types of handheld devices.

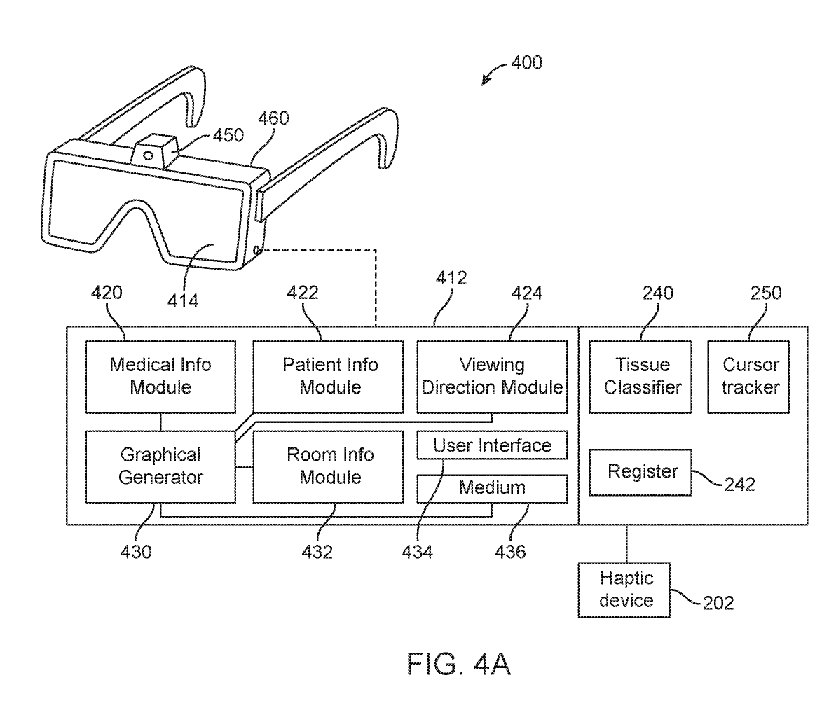

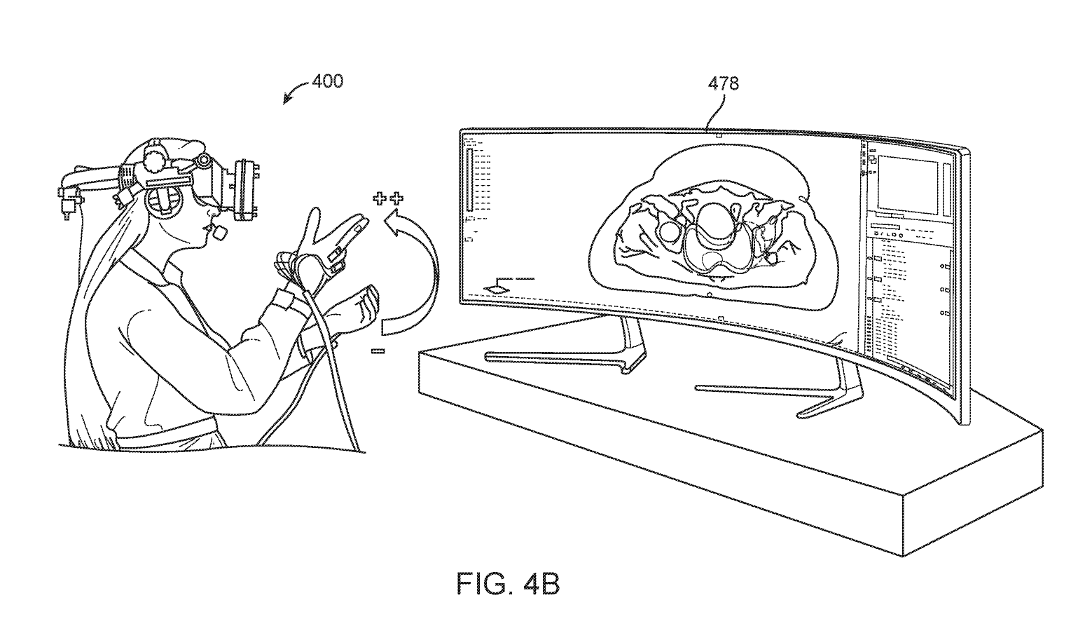

In other embodiments, the screen 182 may be a part of a wearable device. FIG. 4A illustrates an apparatus 400 for use in a medical process that includes a wearable device. The apparatus 400 includes a processing unit 412 and a screen 414 configured for displaying a graphical representation of medical information for a user of the apparatus 400. The processing unit 412 is configured to obtain medical information, obtain a viewing direction of the user of the apparatus, and process the medical information based on the viewing direction of the user of the apparatus 400 to create the graphical representation of the medical information for presentation to the user of the apparatus 400. In some embodiments, the screen 414 may be the screen 182 of FIG. 2. Also, in some embodiments, the processing unit 412 may be the processing unit 210 of FIG. 2.

As shown in the figure, the processing unit 412 of the apparatus 400 includes a medical information module 420 configured to obtain medical information, a patient information module 422 configured to obtain patient information, and a viewing direction module 424 configured to obtain a viewing direction of the user of the apparatus 400. The processing unit 412 also includes a graphics generator 430 coupled to the medical information module 420, the patient information module 422, and the viewing direction module 424. The graphics generator 430 is configured to receive the medical information from the medical information module 420, receive the patient information from the patient information module 422, and the viewing direction from the viewing direction module 424, and create the graphical representation of the medical information for display on the screen 414 of the apparatus 400 for viewing by the user of the apparatus 400.

In the illustrated embodiments, the processing unit 412 also optionally includes a room information module 432 configured to obtain room information. In some cases, the processing unit 412 may create the graphical representation of the medical information also based on the room information from the room information module 432.

The processing unit 412 may also optionally include a user interface 434 configured to receive user input from the user of the apparatus 400. The user interface 434 may be configured to allow a user to enter a command, such as a selection of the type of medical information for display on the screen 414, the format of the graphical representation of the medical information, etc. The user interface 434 may also be configured to receive input from the user for controlling a medical device, such as a treatment planning device, a treatment device, an imaging device, a patient support, or any combination of the foregoing.

The processing unit 412 may also optionally include a non-transitory medium 436 for storing data. The data may be medical information obtained by the medical information module 420, patient information obtained by the patient information module 422, viewing direction obtained by the viewing direction module 424, room information obtained by the room information module 432, or any combination of the foregoing. Also, the data stored in the non-transitory medium may be information derived from the patient information, from the room information, from the viewing direction, or any combination of the foregoing. In some embodiments, the non-transitory medium 436 may also store a treatment plan for a particular patient, and patient identity information for a particular patient. In some embodiments, the non-transitory medium 436 may be the non-transitory medium 220 of FIG. 2.

As shown in FIG. 4A, the apparatus 400 is in a form of a wearable device that includes the screen 414, and a frame 460 to which the screen 414 is secured. In some embodiments, the screen 414 may be transparent (e.g., at least partially transparent) for allowing the user of the apparatus 400 to see the real world (e.g., surrounding environment). The screen 414 may be configured to display the graphics from the graphics generator 430 so that the graphics are superimposed with real objects as directly viewed by the user. Alternatively, the wearable device may be a virtual-reality device. In such cases, the screen 414 is not transparent, and is configured to provide electronic images for viewing by the user. The images may represent the environment around the user, and may be displayed in real-time. Accordingly, the images presented by the electronic screen 414 may change in real time in accordance with a viewing direction of the user.

In other embodiments, the screen 414 may be a part of a holographic device configured to project three-dimensional images in a field of view of the user in real-time.

In some embodiments, the apparatus 400 includes an orientation sensor coupled to the wearable device. For example, the orientation sensor may include one or more accelerometer(s). In such cases, the processing unit 412 may be configured to vary the graphical representation displayed on the screen 414 based on an input from the orientation sensor. For example, as the user of the apparatus 400 tilts or turns his/her head, the processing unit 412 will correspondingly vary the graphics on the screen 414 to match the viewing orientation of the user. Also, in some embodiments, the apparatus 400 includes a positioning device coupled to the wearable device. The positioning device is configured to determine a position of the apparatus 400 with respect to some defined coordinate. The positioning device may use active signals or passive signals to generate positional information regarding a position of the apparatus 400. The processing unit 412 is configured to vary the graphical representation displayed on the screen 414 based on an input from the positioning device. For example, if a user moves further away from the patient, the processing unit 412 will correspondingly vary the graphics (e.g., reduce the size of the graphics) on the screen 414 to match the viewing distance. In further embodiments, the apparatus 400 may include both an orientation sensor and a positioning device. In such cases, the graphical representation displayed on the screen 414 has a variable configuration that corresponds with the viewing direction and viewing distance of the user.

In some embodiments, in addition to the medical information, the processing unit 412 is configured to obtain patient information regarding a geometry of a patient. In such cases, the processing unit 412 may be configured to process the medical information based on both (1) the patient information and (2) the viewing direction of the user of the apparatus 400. By means of non-limiting examples, the patient information may be an image of a person (such as, a digital image of the patient, a digital image of another person different from the patient, or a model of an artificial patient), a size of the patient, a shape of the patient, etc. In some cases, the processing unit 412 may be configured to generate a graphics based on the medical information, and transmit the graphics for display on the screen 414 in a superimposed configuration with respect to the image of the person. In other cases, the patient information may be information regarding a geometry of the patient, and the processing unit 412 may be configured to generate the graphics representing the medical information based on the patient geometry. In one implementation, patient information may be obtained using one or more camera(s). The camera(s) may be optical camera(s), and/or time-of-flight camera(s) configured to provide distance information. The camera(s) may be attached or implemented at the apparatus 400. Alternatively, the camera(s) may be secured to another object (e.g., a wall, a ceiling, a floor, a patient support, a part of a treatment device, etc.) located in a treatment room. In further embodiments, a camera may be attached or implemented at the apparatus 400, while another camera may be secured to another object in the treatment room. In the embodiment in which the camera is a time-of-flight camera, the camera may provide information regarding a surface of the patient that is based on the distance information. In such cases, the output from the camera may be used by the processing unit 412 to generate the surface of the patient, or a model representing a surface of the patient.

In other embodiments, the patient information itself may be considered as an example of medical information.

In further embodiments, the medical information may comprise planned dose, delivered dose, image of internal tissue of a patient, target shape (contour), target position, critical organ shape (contour), critical organ position, contouring of any tissue structure, or any combination of the foregoing. The processing unit 412 is configured to provide a graphics representing such medical information for display on the screen 414, so that the graphics appears in an overlay configuration with respect to the patient, or with respect to an image (e.g., a real-time image) of the patient.

In some embodiments in which the medical information comprises dose information, the processing unit 412 may be configured to create the graphical representation of the dose information based on the viewing direction of the user, and to provide the graphical representation for display over a patient or for display in an overlay configuration with an image of the patient.

Also, in some embodiments, the medical information may comprise tissue geometry (e.g., tissue size, shape, etc.). In such cases, the processing unit 412 may be configured to create the graphical representation of the tissue geometry based on the viewing direction of the user, and to provide the graphical representation for display over a patient or for display in an overlay configuration with an image (e.g., a real-time image) of the patient.

In one or more of the embodiments described herein, the processing unit 412 may be configured to create the graphical representation of the medical information along one or more isocenter axes as viewed by the user. Alternatively, the processing unit 412 may be configured to create the graphical representation of the medical information along a direction that is orthogonal to the viewing direction of the user of the apparatus 400. In further embodiments, the orientation of the graphics representing the medical information may be user-prescribed. In one implementation, the apparatus 400 may include a user interface (e.g., with one or more buttons and/or controls) for allowing the user of the apparatus 400 to select a direction of the cross section of an organ or tissue for display on the screen 414 in an overlay configuration with respect to the patient or with respect to an image (e.g., real-time image) of the patient. For example, if the user wants to see a certain cross section of the liver of the patient while the patient is supported on the patient support, the user may use the user interface of the apparatus 400 to prescribe such cross section with the desired orientation. In such cases, the processing unit 412 will process the user input and derive the cross section based on a CT image of the patient. In some embodiments, the user interface of the apparatus 400 may also allow the user to select which organ or tissue to display on the screen 414.

In other embodiments, the user interface may also allow the user of the apparatus 400 to determine a treatment parameter for a treatment plan while a patient is supported on a patient support. By means of non-limiting examples, the treatment parameter may be a target position to which treatment energy is to be delivered, a critical organ position at which treatment energy is to be limited or avoided, a collision-free zone for protecting the patient (i.e., components of the treatment system cannot move within such collision-free zone), etc.

Also, in some embodiments, the haptic device 202 may be a part of a user control that allows the user to position a cursor displayed on the screen 414 of the wearable device. In one implementation, while internal image of the patient is displayed on the screen 414, the user may operate the user control to move the cursor to different parts of the image.

In addition, in some embodiments, the processing unit 412 may be configured to obtain a CT image of a patient as an example of patient information, and the medical information may be dose information. In such cases, the processing unit 412 may be configured to obtain the medical information by calculating the dose information based on the CT image. For example, one or more anatomical features obtained from the CT image may be utilized in the determination of dose information. The processing unit 412 then generates a graphics representing the dose information for display on the screen 414 of the apparatus 400.

In further embodiments, the processing unit 412 may be configured to obtain a patient model created based on a detected surface of the patient. The detected surface may be obtained using output from one or more time-of-flight cameras (e.g., depth cameras). In such cases, the processing unit 412 may be configured to process the medical information based on the patient model and the viewing direction of the user of the apparatus 400 to create the graphical representation for display on the screen 414 of the apparatus 400. In some cases, the patient model may comprise a volumetric model approximating a shape of the patient and densities within the patient. In one specific example, the patient model may be a CT image, or a cross section of a CT image.

In further embodiments, the medical information may comprise dose information. In such cases, the processing unit 412 may be configured to determine the dose information based on the patient model. For example, the patient model may be used by the process unit 412 to determine certain fiducial point(s) of the patient. The fiducial point(s) establishes certain position and orientation of the patient. Based on the position and orientation of the patient, the processing unit 412 may then create a graphics representing dose information so that the dose information will be aligned with the correct part of the patient (or the correct part of the image of the patient) when the dose information is displayed on the screen 414.

In other embodiments, the medical information may comprise a depth of a treatment isocenter. In such cases, the processing unit 412 may be configured to render the depth of the treatment isocenter over a patient (e.g., with respect to a viewing direction of the user of the apparatus 400), or for display in an overlay configuration with an image (e.g., a real-time image) of the patient.

In some embodiments, the processing unit 412 may also be configured to obtain patient information. For example, the patient information may comprise a position of a patient. Also, the processing unit 412 may obtain image data of the patient as another example of the medical information. In such cases, the processing unit 412 may be configured to create the graphical representation of the image data based on the viewing direction of the user and the position of the patient. The image data may be CT image, ultrasound image, PET image, SPECT image, PET-CT image, MRI image, x-ray image, etc. In some embodiments, if the image data is a CT image, the graphical representation provided by the processing unit 412 may comprise a cross section of a CT image. In one implementation, the processing unit 412 may be configured to create the cross section of the CT image along isocenter axes. Alternatively, the processing unit 12 may be configured to create the cross section of the CT image along a direction that is orthogonal to the viewing direction of the user of the apparatus 400. In some cases, the medical information may also comprise dose information. In such cases, the graphical representation provided by the processing unit 412 may illustrate the dose information on the cross section of the CT image.

As discussed, in some embodiments, the haptic device 202 may be one or more haptic gloves. FIG. 4B illustrates an implementation of the apparatus 400 of FIG. 4A, particularly showing the haptic device being haptic gloves. Also, in some embodiments, instead of displaying graphics on the screen 414 in an overlay configuration with respect to the patient, the user may view towards another screen 478 (a computer screen, flat panel, etc.) to thereby allow the screen 414 of the apparatus 400 to display graphics in an overlay configuration with respect to the screen 478.

The apparatus 400 is advantageous because it allows the user of the apparatus 400 to see internal image of the patient as displayed in an overlay configuration with respect to the patient, or with respect to a real-time image of the patient. This may occur when the user is next to the patient while the patient is positioned next to a treatment device. The user can perform treatment planning task while being next to the patient in the treatment room, and the haptic device 202 will provide mechanical feedback to the user while the user is using a user control to position a cursor over different parts of an image displayed on the screen 414 of the apparatus.

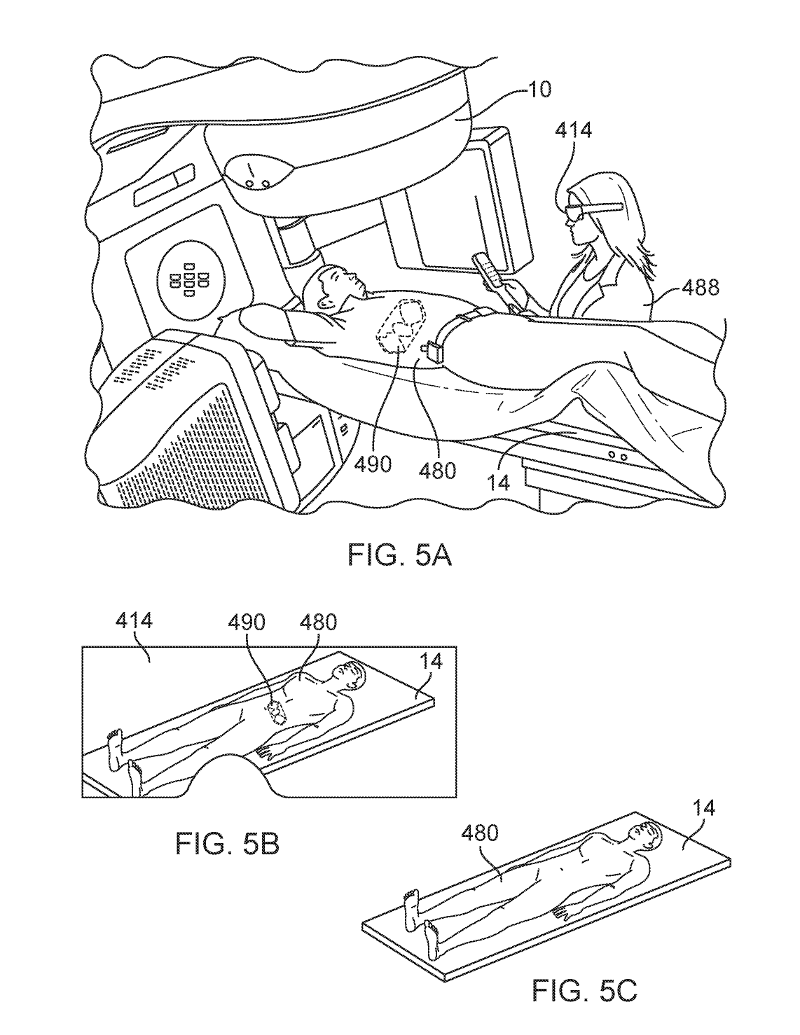

FIGS. 5A-5B illustrate an example of the apparatus 400 providing a graphical representation of medical information in an overlay configuration with respect to a patient 480 or an image (e.g., a real-time image) of the patient 480, while the patient 480 is positioned next to a treatment device. In the illustrated example, the treatment device is the radiation system 10 of FIG. 1. However, in other embodiments, the treatment device may be any of other medical treatment devices. As shown in FIG. 5A, the user 488 is wearing the apparatus 400. The user can see the patient 480 while the patient 480 is being supported on the patient support next to the radiation system 10. The user can also see other objects surrounding the patient via the apparatus 400.

In some embodiments, the screen 414 is transparent, and so the user can see the patient directly through the transparent screen 414. In other embodiments, the screen 414 may be a digital display that is a part of a virtual-reality device. In such cases, the user cannot view through the screen 414 to see the real-world. Instead, the graphics generator 430 may provide images of the patient 480 continuously in real-time. In some cases, the images of the patient 480 may be generated based on signals transmitted from an optical device (e.g., a camera).

Also, as shown in FIG. 5A and FIG. 5B, the user can see medical information 490 as provided by the screen 414 of the apparatus 400. In the illustrated example, the medical information 490 is dose (e.g., delivered dose, predicted dose, and/or planned dose). In such cases, the graphics generator 430 provides a graphical representation of the dose for display on the screen 414, so that when the user view through the screen 414 to see the patient 480, the dose graphics appears in an overlay configuration with respect to the patient 480. As the user moves his/her head to change the viewing direction, the graphical representation of the dose as appeared on the screen 414 will also change correspondingly (e.g., in response to the variable viewing direction of the user). For example, as the user changes the viewing direction to view another part of the patient 480, the graphics generator 430 will correspondingly change the medical information so that the user can see the dose information for the other part of the patient 480. In other cases, the user can view the same part of the patient, but from a different viewing direction. In such cases, the graphical representation of the dose as appeared on the screen 414 will also change correspondingly.

In some embodiments, the dose image as rendered and displayed on the screen 414 of the apparatus 400 may be configurable based on user's preference or selection. For example, a user may use a user interface (e.g., which may be implemented at the apparatus 400, such as one or more buttons at the goggle) to select a direction of rendering for the dose image. In some cases, the user may instruct the processing unit 412 of the apparatus 400 to render the dose image in a direction that is along one or more isocenter axes. In other cases, the user may instruct the processing unit 412 of the apparatus 400 to render the dose image in a direction that is perpendicular to a viewing direction of the user.

As can be seen from the above example, the apparatus 400 is advantageous because it allows the user to see medical information in an overlay configuration with respect to the patient in real-time. This can occur when the user is setting up the patient, reviewing delivered dose after a treatment delivery, setting up the treatment machine for a next treatment delivery, reviewing a treatment plan, and/or adjusting the treatment plan. Without the apparatus 400, the user can only see the patient 480, and there is no medical information available for the user to view while the user is looking at the patient 480 (FIG. 5C).

In the example shown in FIG. 5A, there is only one user wearing the apparatus 400. In other embodiments, there may be multiple users wearing corresponding apparatuses 400.

In the above example, the dose information may be considered to be an example of medical information. In other example, the medical information may be image data of the patient. By means of non-limiting examples, the image data may be CT image, digital x-ray image, ultrasound image, MRI image, PET image, PET-CT image, SPECT image, SPECT-CT image, etc.

In some cases, when image data is displayed on the screen 414, the user may utilize the user control to perform contouring, segmentation, dose painting, any of other treatment planning tasks, or any combination of the foregoing, on the image while the image is being displayed in an overlay configuration with respect to the patient 480 or with respect to a real-time image of the patient 480. In the example shown in FIG. 5A, the user control is a hand-held control that includes the haptic device 202. The haptic device 202 provides mechanical feedback to the user as the user operates the user control to position a cursor (displayed on the screen 414) to different parts of the image of the patient.

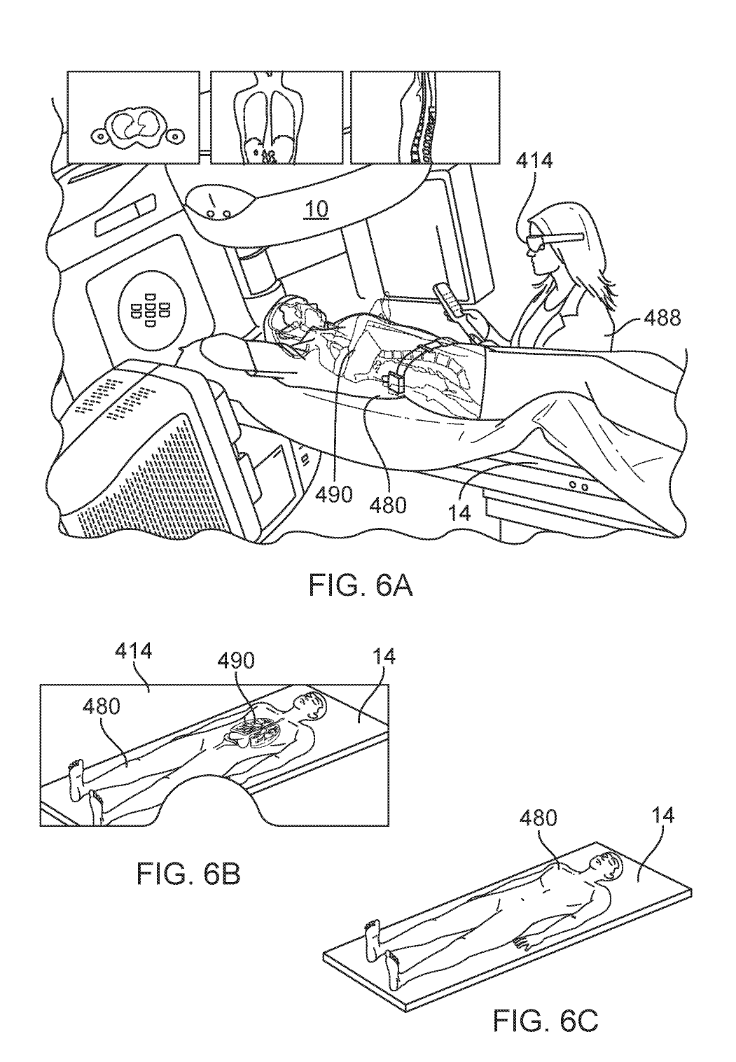

FIGS. 6A-6B illustrates another example of the apparatus 400 providing a graphical representation of medical information in an overlay configuration with respect to a patient or an image (e.g., a real-time image) of the patient, while the patient is positioned next to a treatment device. In the illustrated example, the treatment device is the radiation system 10 of FIG. 1. However, in other embodiments, the treatment device may be any of other medical treatment devices. As shown in FIG. 6A, the user is wearing the apparatus 400. The user can see the patient 480 while the patient 480 is being supported on the patient support next to the radiation system 10. The user can also see other objects surrounding the patient 480 via the apparatus 400.

Also, as shown in FIG. 6A and FIG. 6B, the user can see medical information 490 as provided by the screen 414 of the apparatus 400. In the illustrated example, the medical information 490 is internal image (CT image) of the patient 480. In such cases, the graphics generator 430 provides the internal image for display on the screen 414, so that when the user view through the screen 414 to see the patient 480, the internal image appears in an overlay configuration with respect to the patient 480. As the user moves his/her head to change the viewing direction, the internal image as appeared on the screen 414 will also change correspondingly (e.g., in response to the variable viewing direction of the user). For example, as the user changes the viewing direction to view another part of the patient 480, the graphics generator 430 will correspondingly change the medical information so that the user can see the internal image for the other part of the patient 480. In other cases, the user can view the same part of the patient, but from a different viewing direction. In such cases, the internal image of the patient 480 as appeared on the screen 414 will also change correspondingly.

In some embodiments, the CT image as rendered and displayed on the screen 414 of the apparatus 400 may be configurable based on user's preference or selection. For example, a user may user a user interface (e.g., which may be implemented at the apparatus 400, such as one or more buttons at the goggle) to select a direction of rendering for the CT image. In some cases, the user may instruct the processing unit 412 of the apparatus 400 to render the CT image in a direction that is along one or more isocenter axes. In other cases, the user may instruct the processing unit 412 of the apparatus 400 to render the CT image in a direction that is perpendicular to a viewing direction of the user. Also, the user may instruct the processing unit 412 to provide surface rendering, which shows organ surfaces. In other cases, the user may instruct the processing unit 412 to provide cross sectional view of the internal organs of the patient 480.

In the above example, the medical information is image data that comprises CT image. In other embodiments, the image data may be digital x-ray image, ultrasound image, MRI image, PET image, PET-CT image, SPECT image, SPECT-CT image, etc.

As can be seen from the above example, the apparatus 400 is advantageous because it allows the user to see medical information 490 in an overlay configuration with respect to the patient in real-time. This can occur when the user is setting up the patient, reviewing delivered dose after a treatment delivery, setting up the treatment machine for a next treatment delivery, reviewing a treatment plan, and/or adjusting the treatment plan. Without the apparatus 400, the user can only see the patient 480, and there is no medical information available for the user to view while the user is looking at the patient 480 (FIG. 6C).

In some cases, when image data is displayed on the screen 414, the user may utilize the user control to perform contouring, segmentation, dose painting, any of other treatment planning tasks, or any combination of the foregoing, on the image while the image is being displayed in an overlay configuration with respect to the patient 480 or with respect to a real-time image of the patient 480. In the example shown in FIG. 6A, the user control is a hand-held control that includes the haptic device 202. The haptic device 202 provides mechanical feedback to the user as the user operates the user control to position a cursor (displayed on the screen 414) to different parts of the image of the patient.

In the example shown in FIG. 6A, there is only one user wearing the apparatus 400. In other embodiments, there may be multiple users wearing corresponding apparatuses 400.

In one or more embodiments described herein, the processing unit 412 is configured to align the graphics as displayed on the screen 414 with a certain part of the patient, or with a certain part of an image of the patient. This way, as the user of the apparatus 400 changes his/her viewing direction, the graphics will change in real-time and will remain aligned with the correct part of the patient or the correct part of the image of the patient. In one implementation, the apparatus 400 may be configured to detect certain part(s) of the patient in real-time. Such may be accomplished using one or more cameras to view the patient. Images from the camera(s) may then be processed by the processing unit 412 to determine the position(s) of certain part(s) of the patient. In some cases, markers may be placed at the patient to facilitate the accomplishment of such purpose. In other cases, anatomical landmarks at the patient may be utilized as markers. In other embodiments, the camera(s) may be depth camera(s) for detecting the surface of the patient. The detected surface may then be utilized by the processing unit 412 to identify the position of the patient (e.g., position(s) of certain part(s) of the patient). Once the actual position of the certain part(s) of the patient has been determined, the processing unit 412 then determines a position of the graphics (representing certain medical information) with respect to the determined actual position. The position of the graphics may then be utilized by the processing unit 412 for correct positioning of the graphics at the right location of the screen 414. For example, if the medical information comprises an image of an internal part of the patient, the position of the internal part of the patient with respect to certain part P of the patient is known, or may be derived from analysis of the image. During use of the apparatus 400, the processing unit 412 analyzes real-time images of the patient to determine the actual position of the same part P of the patient. Based on the known relative positioning between the image of the internal part of the patient and the certain part P of the patient, then processing unit 412 then places the graphics (representing the same internal part of the patient) at the same relative position with respect to the actual position of the certain part P of the patient at the screen 414 in real-time.

It should be noted that the apparatus 400 is not limited to a wearable device that is in a form of goggle or glasses. In other embodiments, the apparatus 400 may be in a form of a helmet, hood, facemask, etc., that is for worn at the head of the user.

In the above embodiments, the apparatus 400 was described as being used next to the patient while the patient is supported on a patient support next to the treatment system 10. In some cases, the user may utilize the apparatus 400 to perform patient setup. Also, the user may utilize the apparatus 400 to perform treatment planning task(s) before the treatment system 10 delivers treatment energy towards the patient. The user may also utilize the apparatus 400 to perform treatment planning task(s) between deliveries of treatment energies while the patient is being supported on the patient support next to the treatment system 10. In other embodiments, the apparatus 400 is not limited to being used next to the patient while the patient is supported on the patient support next to the treatment system 10. For example, the apparatus 400 may be used by a user to perform treatment planning on a different day from the treatment day. The treatment planning may be performed on the patient while the patient is supported on a patient support next to an imaging device. Alternatively, the treatment planning may be performed on a phantom.

Also, the apparatus 400 is not required to have all of the above features described herein. In other embodiments, one or more of the features described may not be included with the apparatus 400.

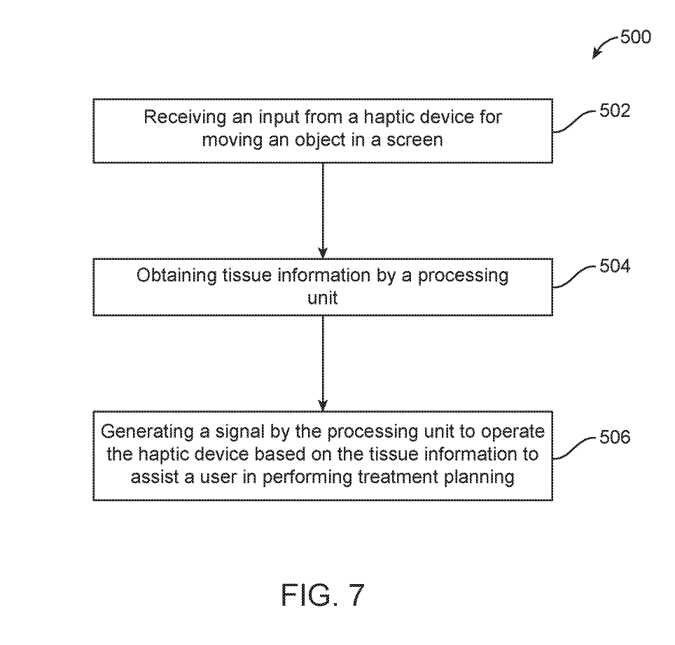

FIG. 7 illustrates a method 500 in accordance with some embodiments. The method 500 may be performed for treatment planning. The method 500 includes receiving an input from a haptic device for moving an object in a screen (item 502); obtaining tissue information by a processing unit (item 504); and generating a signal by the processing unit to operate the haptic device based on the tissue information to assist a user in performing treatment planning (item 506). In some embodiments, the method 500 may be performed by the apparatus 200 or by the apparatus 400.

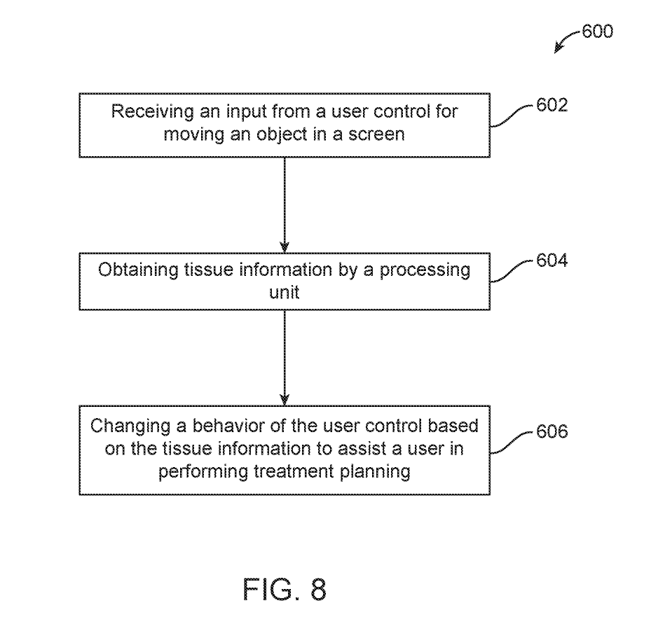

FIG. 8 illustrates a method 600 in accordance with some embodiments. The method 600 may be performed for treatment planning. A method 600 includes: receiving an input from a user control for moving an object in a screen (item 602); obtaining tissue information by a processing unit (item 604); and changing a behavior of the user control based on the tissue information to assist a user in performing treatment planning (item 606). In some embodiments, the method 600 may be performed by the apparatus 200 or by the apparatus 400.

Specialized Processing System

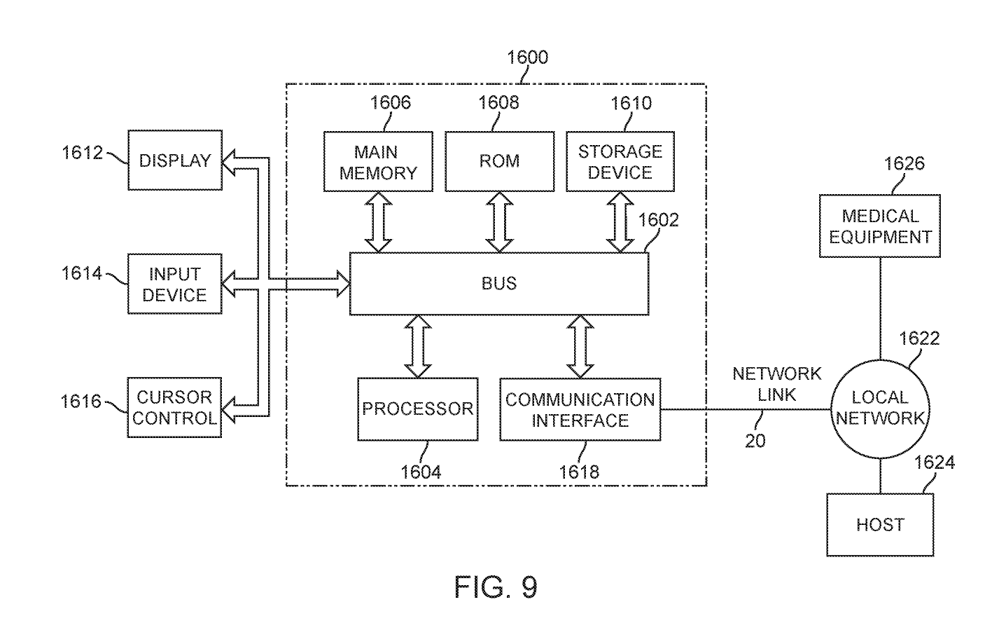

FIG. 9 is a block diagram illustrating an embodiment of a specialized processing system 1600 that can be used to implement various embodiments described herein. For example, the processing system 1600 may be configured to provide one, some, or all of the functions of the apparatus 200/400 in accordance with some embodiments. Also, in some embodiments, the processing system 1600 may be used to implement the processing unit 210, the processing unit 412, and/or the processing unit 54. The processing system 1600 may also be an example of any processor described herein. Furthermore, the processing system 1600 may be configured to perform the method 500 of FIG. 7 and/or the method 600 of FIG. 8.

Processing system 1600 includes a bus 1602 or other communication mechanism for communicating information, and a processor 1604 coupled with the bus 1602 for processing information. The processor system 1600 also includes a main memory 1606, such as a random access memory (RAM) or other dynamic storage device, coupled to the bus 1602 for storing information and instructions to be executed by the processor 1604. The main memory 1606 also may be used for storing temporary variables or other intermediate information during execution of instructions to be executed by the processor 1604. The processor system 1600 further includes a read only memory (ROM) 1608 or other static storage device coupled to the bus 1602 for storing static information and instructions for the processor 1604. A data storage device 1610, such as a magnetic disk or optical disk, is provided and coupled to the bus 1602 for storing information and instructions.

The processor system 1600 may be coupled via the bus 1602 to a display 167, such as a cathode ray tube (CRT), for displaying information to a user. An input device 1614, including alphanumeric and other keys, is coupled to the bus 1602 for communicating information and command selections to processor 1604. Another type of user input device is cursor control 1616, such as a mouse, a trackball, or cursor direction keys for communicating direction information and command selections to processor 1604 and for controlling cursor movement on display 167. This input device typically has two degrees of freedom in two axes, a first axis (e.g., x) and a second axis (e.g., y), that allows the device to specify positions in a plane.

In some embodiments, the processor system 1600 can be used to perform various functions described herein. According to some embodiments, such use is provided by processor system 1600 in response to processor 1604 executing one or more sequences of one or more instructions contained in the main memory 1606. Those skilled in the art will know how to prepare such instructions based on the functions and methods described herein. Such instructions may be read into the main memory 1606 from another processor-readable medium, such as storage device 1610. Execution of the sequences of instructions contained in the main memory 1606 causes the processor 1604 to perform the process steps described herein. One or more processors in a multi-processing arrangement may also be employed to execute the sequences of instructions contained in the main memory 1606. In alternative embodiments, hard-wired circuitry may be used in place of or in combination with software instructions to implement the various embodiments described herein. Thus, embodiments are not limited to any specific combination of hardware circuitry and software.

The term “processor-readable medium” as used herein refers to any medium that participates in providing instructions to the processor 1604 for execution. Such a medium may take many forms, including but not limited to, non-volatile media, volatile media, and transmission media. Non-volatile media includes, for example, optical or magnetic disks, such as the storage device 1610. A non-volatile medium may be considered an example of non-transitory medium. Volatile media includes dynamic memory, such as the main memory 1606. A volatile medium may be considered an example of non-transitory medium. Transmission media includes coaxial cables, copper wire and fiber optics, including the wires that comprise the bus 1602. Transmission media can also take the form of acoustic or light waves, such as those generated during radio wave and infrared data communications.

Common forms of processor-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a processor can read.

Various forms of processor-readable media may be involved in carrying one or more sequences of one or more instructions to the processor 1604 for execution. For example, the instructions may initially be carried on a magnetic disk of a remote computer. The remote computer can load the instructions into its dynamic memory and send the instructions over a telephone line using a modem. A modem local to the processing system 1600 can receive the data on the telephone line and use an infrared transmitter to convert the data to an infrared signal. An infrared detector coupled to the bus 1602 can receive the data carried in the infrared signal and place the data on the bus 1602. The bus 1602 carries the data to the main memory 1606, from which the processor 1604 retrieves and executes the instructions. The instructions received by the main memory 1606 may optionally be stored on the storage device 1610 either before or after execution by the processor 1604.

The processing system 1600 also includes a communication interface 1618 coupled to the bus 1602. The communication interface 1618 provides a two-way data communication coupling to a network link 1620 that is connected to a local network 1622. For example, the communication interface 1618 may be an integrated services digital network (ISDN) card or a modem to provide a data communication connection to a corresponding type of telephone line. As another example, the communication interface 1618 may be a local area network (LAN) card to provide a data communication connection to a compatible LAN. Wireless links may also be implemented. In any such implementation, the communication interface 1618 sends and receives electrical, electromagnetic or optical signals that carry data streams representing various types of information.

The network link 1620 typically provides data communication through one or more networks to other devices. For example, the network link 1620 may provide a connection through local network 1622 to a host computer 1624 or to equipment 1626 such as a radiation beam source or a switch operatively coupled to a radiation beam source. The data streams transported over the network link 1620 can comprise electrical, electromagnetic or optical signals. The signals through the various networks and the signals on the network link 1620 and through the communication interface 1618, which carry data to and from the processing system 1600, are exemplary forms of carrier waves transporting the information. The processing system 1600 can send messages and receive data, including program code, through the network(s), the network link 1620, and the communication interface 1618.

Although particular embodiments have been shown and described, it will be understood that it is not intended to limit the claimed inventions to the preferred embodiments, and it will be obvious to those skilled in the art that various changes and modifications may be made without department from the spirit and scope of the claimed inventions. The specification and drawings are, accordingly, to be regarded in an illustrative rather than restrictive sense. The claimed inventions are intended to cover alternatives, modifications, and equivalents.

1. An apparatus for use in a medical process, comprising:
a haptic device configured to provide mechanical feedback to a user; and
a processing unit communicatively coupled to the haptic device, wherein the processing unit is configured to obtain tissue information, and provide a signal to operate the haptic device based on the tissue information for assisting the user in performing treatment planning.

2. The apparatus of claim 1, further comprising a non-transitory medium storing movement-vs-intensity profiles for different types of tissue, wherein the tissue information is associated with one of the types of tissue.

3. The apparatus of claim 2, wherein one of the movement-vs-intensity profiles indicates how an intensity of force resistance, or an intensity of vibration, changes with user movement.

4. The apparatus of claim 2, wherein the different types of tissue comprise two or more of bladder, spine, liver, kidney, cochlea, target, and critical organ.

5. The apparatus of claim 1, wherein the haptic device is configured to provide force resistance as the mechanical feedback.

6. The apparatus of claim 5, wherein an intensity of the force resistance is variable in correspondence with the tissue information.

7. The apparatus of claim 1, wherein the haptic device is configured to provide vibration as the mechanical feedback.

8. The apparatus of claim 7, wherein an intensity of the vibration is variable in correspondence with the tissue information.

9. The apparatus of claim 1, wherein the tissue information comprises a type of tissue, a position of the tissue, clinical information about the tissue, a radiological property of the tissue, or any combination of the foregoing.

10. The apparatus of claim 1, wherein the haptic device comprises a stick for held by the user.

11. The apparatus of claim 1, wherein the haptic device comprises a mouse.

12. The apparatus of claim 1, wherein the haptic device comprises a touch screen.

13. The apparatus of claim 1, wherein the processing unit is configured to provide the feedback for assisting the user in performing structure contouring.

14. The apparatus of claim 1, wherein the processing unit is configured to provide the feedback for assisting the user in performing dose painting.

15. The apparatus of claim 1, further comprising a wearable device with a screen, the screen being communicatively coupled to the processing unit.

16. The apparatus of claim 15, further comprising an orientation sensor coupled to the wearable device, wherein the processing unit is configured to vary an object displayed on the screen based on an input from the orientation sensor.

17. The apparatus of claim 15, further comprising a positioning device coupled to the wearable device, wherein the processing unit is configured to vary an object displayed on the screen based on an input from the positioning device.

18. The apparatus of claim 15, wherein the wearable device comprises a virtual-reality device.

19. The apparatus of claim 15, wherein the screen comprises a transparent screen for allowing the user to see surrounding space.

20. The apparatus of claim 1, further comprising a device with a screen, the screen being communicatively coupled to the processing unit.

21. The apparatus of claim 20, wherein the screen is a part of a handheld device.

22. The apparatus of claim 20, wherein the processing unit is configured to cause the screen to display an object, and to vary a configuration of the object in correspondence with a viewing direction of the user.

23. An apparatus for use in a medical process, comprising:
a feedback device configured to provide visual feedback to a user; and
a processing unit communicatively coupled to the feedback device;
wherein the visual feedback comprises a displayed object, wherein a position of the displayed object is variable in response to operation of a user control, and wherein the processing unit is configured to obtain tissue information, and to change a behavior of the user control based on the tissue information.

24. The apparatus of claim 23, wherein the feedback comprises a screen.

25. The apparatus of claim 23, further comprising a non-transitory medium storing movement-vs-intensity profiles for different types of tissue, wherein the tissue information is associated with one of the types of tissue.

26. The apparatus of claim 25, wherein one of the movement-vs-intensity profiles indicates how an intensity of force resistance, or an intensity of vibration, changes with user movement.

27. The apparatus of claim 25, wherein the different types of tissue comprise two or more of bladder, spine, liver, kidney, cochlea, target, and critical organ.

28. The apparatus of claim 23, wherein the tissue information comprises a type of tissue, a position of the tissue, clinical information about the tissue, a radiological property of the tissue, or any combination of the foregoing.

29. The apparatus of claim 23, wherein the operation of the user control is for performing structure contouring.

30. The apparatus of claim 23, wherein the operation of the user control is for performing dose painting.

31. The apparatus of claim 23, wherein the processing unit is configured to change the behavior of the user control by changing an amount of movement of the displayed object per unit of user movement on the user control.

32. A method for treatment planning, comprising:
receiving an input from a haptic device for moving an object in a screen;
obtaining tissue information by a processing unit; and
generating a signal by the processing unit to operate the haptic device based on the tissue information to assist a user in performing treatment planning.

33. A method for treatment planning, comprising:
receiving an input from a user control for moving an object in a screen;
obtaining tissue information by a processing unit; and
changing a behavior of the user control based on the tissue information to assist a user in performing treatment planning.
